(19) United States
(12) Patent Application Publication
(10) Pub. No.: US 2016/0253463 A1
(43) Pub. Date: Sep. 1, 2016

(54) SIMULATION-BASED SYSTEMS AND METHODS TO HELP HEALTHCARE CONSULTANTS AND HOSPITAL ADMINISTRATORS DETERMINE AN OPTIMAL HUMAN RESOURCE PLAN FOR A HOSPITAL

(71) Applicant: KONINKLIJKE PHILIPS N.V., Eindhoven (NL)

(72) Inventors: ZHICHAO SHU, Briarcliff Manor, NY (US); JINGYU ZHANG, Elmsford, NY (US); XIANG ZHONG, Briarcliff Manor, NY (US)

(21) Appl. No.: 15/049,267

(22) Filed: Feb. 22, 2016

Related U.S. Application Data
(60) Provisional application No. 62/121,558, filed on Feb. 27, 2015.

(51) Int. Cl. G06F 19/00

(52) U.S. Cl. CPC G06F 19/327

(57) ABSTRACT

A method 200 for creating a human resources plan for a hospital system is provided. At Step 202, one or more inputs 46, 48, 50 related to one or more health care services that are each associated with at least one of hospital data and target data are received. At Step 204, variations of the one or more inputs 46, 48, 50 are simulated. At Step 206, the one or more inputs 46, 48, 50 are optimized from the simulated input variations. At Step 208, one or more output human resource plans 78 are created from the optimized inputs.

### FIELD

The following relates generally to systems and methods for creating optimized human resource (HR) plans for a hospital system. It finds particular application in conjunction with systems and methods for optimizing one or more parameters of hospital and patient data to generate human resource plans for a hospital system and will be described with particular reference thereto. However, it is to be understood that it also finds application in other usage scenarios and is not necessarily limited to the aforementioned application.

### BACKGROUND

Human resource (HR) planning is of great importance in healthcare area, especially for newly opened hospitals, but also for existing hospitals, e.g. to plan expansions, account for demographic changes in the served population, and so forth. An understaffed hospital is less effective at treating patients, while overstaffing leads to excessive human resource expenditures that might be better used for equipment upgrades, additional beds, or the like. Staffing is a challenging task because it is not simply a question of having enough employees, but also having employees with appropriate medical specializations, experience and expertise levels.

Current approaches for human resource planning typically determine target human resource levels based on benchmark data, predicted patient volume data, and other information such as average patient visit time and average surgery/procedure time. These data sets are treated as fixed values and variations are typically not taken into account in their calculation.

Hospitals are also subject to a complex web of regulations. In the United States, a hospital may be subject to Federal, state, county, and city regulations, in diverse areas such as medical, employment, and physical infrastructure. Similarly complex regulatory networks exist in many other countries. Patient demographics also vary widely: a hospital in one area may see mostly cardiac cases, while a hospital in another area may see few cardiac cases but many cases in other areas. Due to different regulations and requirements in hospitals and the complexity of patient related variations in healthcare system, healthcare consultants and hospital administrators could benefit from an effective analytic tool to assist in human resource planning.

### SUMMARY

The present disclosure provides new and improved systems and methods which overcome the above-referenced problems and others.

This present disclosure aims to support the creation of an optimized human resource plan to address resources allocation and use for treating patients in a hospital. The present disclosure provides system and methods to: (1) model and simulate variations in healthcare system including patient arrivals, patient visit time, surgery/procedure time, etc.; (2) determine the optimal number of different types of healthcare employees working in different units in a hospital based on a pre-defined multi-goal objective function while satisfying certain regulations and requirements; (3) provide more comprehensive outputs such as coverage rate, utility and average overtime based on the optimal full-time equivalent (FTE) numbers and the simulation model; (4) and utilize sensitivity analysis to find which parameters have the most influences on the outputs.

In accordance with one aspect, a human resources (HR) planning system comprises an electronic processor programmed to perform a HR planning method including:

generating a tentative HR plan based on received parameters including at least patient volume parameters and staffing parameters for a plurality of HR specialty units; computing a simulated HR plan from the tentative HR plan based on received parameter variability data, the simulated HR plan representing parameters of the tentative HR plan as random variables with distributions representing the parameter variability; optimizing the random variables of the simulated HR plan with respect to an objective function representing objectives for staffing of the medical institution; and outputting staffing plans for the HR specialty units wherein the staffing plans are determined from the optimized random variables representing the staffing parameters in the optimized simulated HR plan.

In accordance with another aspect, a non-transitory storage medium stores instructions readable and executable by an electronic processor to perform a human resources (HR) planning method comprising: generating a tentative HR plan based on received parameters including patient volume parameters and staffing parameters for a plurality of specialty units defined at least by medical expertise into physician, nursing, and non-clinical support staff specialty units; computing a simulated HR plan from the tentative HR plan based on received parameter variability data, the simulated HR plan representing parameters of the tentative HR plan as random variables with distributions representing the parameter variability; performing a constrained optimization of the random variables of the simulated HR plan with respect to an objective function representing objectives for staffing of the medical institution and constraints defined at least by governmental regulations; and outputting staffing plans for the specialty units wherein the staffing plans are determined from the optimized random variables representing the staffing parameters in the optimized simulated HR plan.

In accordance with another aspect, a method for creating a human resources plan for a hospital system is provided. One or more inputs related to one or more health care services that are each associated with at least one of hospital data and target data are received at an electronic processor. Variations of the one or more inputs are simulated. The one or more inputs are optimized from the simulated input variations. One or more output human resource plans are created from the optimized inputs. The simulating, optimizing, and creating are suitably performed by the electronic processor.

One advantage resides in providing an analytic tool that generates a human resources plan by leveraging input data that captures statistical information, suitably represented as random variables.

Another advantage resides in creating a human resources plan with more comprehensive outputs, including statistical information such as confidence intervals or uncertainty estimates for the parameters.

Another advantage resides in creating a human resources plan that minimizes costs while satisfying hospital requirements and regulations.

Another advantage resides in creating a human resources plan which allows the determination of optimal parameters for future data collection.

Still further advantages of the present disclosure will be appreciated to those of ordinary skill in the art upon reading and understanding the following detailed description. It is to be understood that a given embodiment may achieve none, one, two, more, or all of these advantages.

### BRIEF DESCRIPTION OF DRAWINGS

The disclosure may take form in various components and arrangements of components, and in various steps and arrangements of steps. The drawings are only for purposed of illustrating the preferred embodiments and are not to be construed as limiting the disclosure.

FIG. 1 is a schematic view showing a human resources planning system in accordance with one aspect of the present disclosure;

FIG. 2 is a schematic view showing multiple components of the human resource plan system of FIG. 1;

FIG. 3 is an exemplary flow chart of one example use of the patient care plan system of FIG. 1;

FIG. 4 is a schematic view showing another example use of the patient care plan system of FIG. 1;

FIG. 5 is a tabular view showing data of one input associated with the human resource plan system of FIG. 1;

FIG. 6 is a tabular view showing data of one output associated with the human resource plan system of FIG. 1; and

FIGS. 7A and 7B are graphical views showing multiple outputs associated with the human resource plan system of FIG. 1.

### DETAILED DESCRIPTION

Current human resource (HR) planning tools typically treat all input parameters as fixed values and thus cannot capture the existing variations in healthcare system. In human resources planning tools disclosed herein, parameters are considered as random variables to allow users to specify distributions (i.e. statistics) for the parameters as input data. Current human resource planning tools typically only report full time equivalent (FTE) numbers as output. FTE is a conventional unit that expresses the workload (and hence the target workforce) in terms of an equivalent number of full-time employees (although the workload might be able to be handled by a greater number of workers than the FTE with some workers being part-time, or by fewer workers who put in some overtime). No further statistics are typically provided. However, healthcare consultants and hospital administrators engaged in human resources planning may want to have additional information to help them understand the outcomes that can be expected for various staffing options. Human resources planning tools disclosed herein provide more comprehensive outputs, such as coverage rate, utility and average overtime, and provide the capability of mathematical optimization in order to minimize the total cost of the staffing plan while still satisfying applicable hospital requirements and regulations. Human resources planning tools disclosed herein further allow a user to determine which parameters of the staffing plan are most important and in this way facilitate prioritizing further data collection. Sensitivity analysis is provided which provides decision makers with information about which parameters are more important so as to prioritize further data collection.

The present disclosure is directed to systems and methods for creating optimized human resource plans for a hospital system. As discussed in more detail below, the systems and methods of the present disclosure provide optimizing one or more parameters of hospital and patient data to generate human resource plans for a hospital system. The present disclosure provides a human resource plan to build variations and evaluate plans using simulations to find the optimal human resource plan instead of calculating the FTE numbers based on known benchmark ratio. Advantageously, the systems and methods of the present disclosure provide a processor that: (1) models and simulates variations in healthcare system (such as patient arrivals, patient visit time, surgery/procedure time, and the like); (2) determines the optimal number of different types of healthcare employees working in different units in a hospital based on a pre-defined multi-goal objective function while satisfying certain regulations and requirements; (3) provides more comprehensive outputs (such as coverage rate, utility, average overtime, and the like) based on optimal FTE numbers and a simulation model; and (4) utilizes a sensitivity analysis to find parameters that have the most influences on an output human resource health care plan.

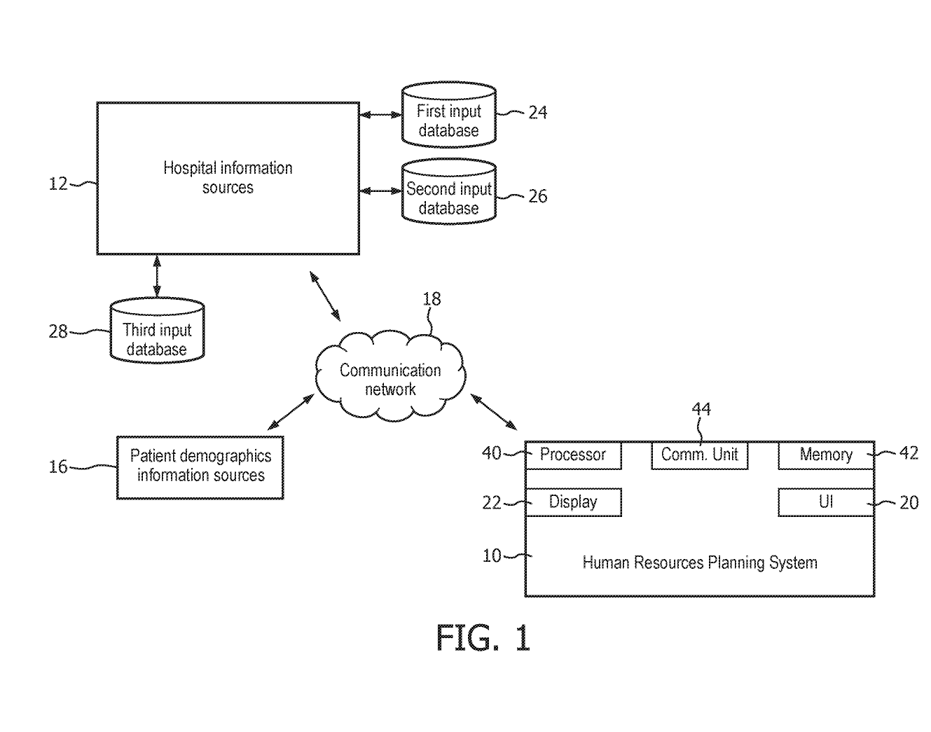

With reference to FIG. 1, a block diagram illustrates one embodiment of a human resources planning system 10 for predicting and optimizing the human resources (i.e. staffing) requirements for a medical institution, such as a hospital. The human resources planning system 10 may be operative before or during construction of the hospital (in the case of a newly built hospital) in order to plan initial staffing needs and to project likely changes (e.g. growth) in staffing needs over time, and/or may be operative to provide assistance in human resources planning for an existing, operating hospital, for example to account for an expansion of provided services, to plan for changing patient demographics, to deal with anticipated changes in hospital funding, or so forth. The human resources planning system 10 may utilize data from various sources, such hospital information sources 12 and patient demographics information sources 16. The hospital information sources may be specific to the hospital that is the target of the planning, if it is already in operation, or may be information sources for similarly situated hospitals in the case of human resources planning for a new hospital. The human resources planning system 10 may acquire data via a communications network 18. It is contemplated that the communications network 18 includes one or more of the Internet, Intranet, a local area network, a wide area network, a wireless network, a wired network, a cellular network, a data bus, and the like. Additionally or alternatively, information for the human resources planning may be provided in other ways, such as by manual input to a computer implementing the human resources planning system 10, loading data into the system 10 using a physical medium such as an optical disk storing the data, or so forth.

Various data may be gathered from the sources 12, 16. In some examples, the human resources-related data can be gathered automatically (e.g. via the electronic data network 18) and/or manually. To gather the data manually, one or more user input devices 20 can be employed (e.g. a keyboard, mouse, or so forth), with the data entry operator viewing a display device 22 of the human resources planning system 10 that provides users a user interface within which to manually enter the human resource data and/or for displaying generated human resource data. By way of illustration, human resource-related data that may be input to the system 10 includes: (1) benchmark data (e.g., information related to staffing buffer, patient to nurse ratio, bed occupancy rate from literature or other hospitals, and the like); (2) patient volume data (e.g., distributions of ambulatory and inpatient visits for different specialty units which can be fitted from historical data, and the like); (3) specialty procedure information data (e.g., distributions of ambulatory visit time, inpatient ward time, patient length of stay for different specialty units which can be fitted from historical data, and the like); (4) miscellaneous general data (e.g., working hours per day, working days within a year, percentage of patient related activity, and the like); (5) regulations and requirements data (e.g., minimum coverage rate, maximum overtime, percentage of senior/staff/assistant nurse, and the like); and (6) multi-goal objective function data (e.g., several different goals and the corresponding weights minimized by the weighted sum of total FTE number and average overtime). In the case of hospital information sources 12, the human resource-related data is stored in one or more hospital information databases 24, 26, 28, such as electronic medical record systems, departmental systems, and the like.

The hospital information sources 16 may include information sources pertaining to benchmark data, the patient volume data, the specialty procedure information data, as well as applicable governmental regulations and requirements data, and the multi-goal objective function data. In one embodiment, the user interface system of the human resources planning system 10 enables the user to enter specific settings for human resource data. These settings may include FTE values, available medical equipment, available medical staff, and the like. The user interface system includes the display 22 (such as a CRT display, a liquid crystal display, a light emitting diode display, and the like) to display the evaluation and/or comparison of choices and the user input device 20 such as a keyboard and a mouse, for the user to input the patient values and preferences and/or modify the evaluation and/or comparison.

The components of the human resources planning system 10 suitably include one or more electronic processors 40 executing computer executable instructions embodying the foregoing functionality, where the computer executable instructions are stored on memories 42 and/or on a hard disk drive, optical disk, or other non-transitory storage media 42 associated with the processors 40. Further, the components of the illustrative human resources planning system 10 include communication units 44 providing the one or more processors 40 an interface from which to communicate over the communications network 18. Even more, although the foregoing components of the human resource plan system 10 were discretely described, it is to be appreciated that the components can be variously combined.

The human resources planning processor 10 is associated with each of the first human resource information database 24, the second human resource information database 26, and the third human resource information database 28. The human resource planning processor 10 includes a tentative human resource plan processor 52, a simulation processor (i.e. variation processing unit) 54, and an optimization processor 56. The tentative human resource plan processor 52 is programmed to generate a tentative human resource plan 58 based on the set of first inputs 46. For example, the tentative human resource plan processor 52 generates the tentative human resource plan 58 based on the benchmark data (e.g., information related to staffing buffer, patient to nurse ratio, bed occupancy rate from literature or other hospitals, and the like). To do so, the tentative human resource plan processor 52 includes a data mining processor 60 programmed to extract values related to the benchmark data (e.g., FTE values, distribution data, and the like) from the first human resource information database 24. For example, the set of first inputs 46 is input into a pre-calculated lookup table, a neural network, or the like. The tentative human resource plan processor 52 also includes a tentative plan generator processor 62 programmed to generate a tentative human resource plan 5858 reflecting a general view of the hospital resources. The tentative plan processor 62 uses metaheuristic methods (e.g., a greedy algorithm, a Tabu search, a genetic algorithm, simulated annealing, and the like) along with inputs from the set of first inputs 46 to create the tentative human resource plan 5858.

The simulation processor 54 is programmed to model variations in data of the set of second inputs 48 and/or the tentative human resource plan 58. To do so, the simulation processor 54 includes a data mining processor 66 programmed to extract value related to the patient volume data, the specialty procedure information data, and the miscellaneous general data. Similarly, the data mining processor 66 is also programmed to extract similar values from the tentative human resource plan 58. For example, the set of second inputs 48 and/or the general human resource plan 58 is input into a pre-calculated lookup table, a neural network, or the like. The simulation processor 54 is programmed to generate random numbers from distributions specified in the data of the set of the second inputs 48 and the tentative human resource plan 58. The simulation processor 54 also includes a simulated plan generator processor 68 programmed to generate a simulated human resource plan 70 reflecting a simulated view of the hospital resources. The simulated plan processor 68 uses metaheuristic methods (e.g., a greedy algorithm, a Tabu search, a genetic algorithm, simulated annealing, and the like) along with inputs from the set of second inputs 48 and the tentative human resource plan 58 to create the simulated human resource plan 70.

The optimization processor 56 is programmed to model variations in data of the set of third inputs 50 and/or the simulated human resource plan 70. To do so, the optimization processor 56 includes a data mining processor 72 programmed to extract values related to the regulations and requirements data and the multi-goal objective function data. Similarly, the data mining processor 72 is also programmed to extract similar values from the simulated human resource plan 70. For example, the set of third inputs 50 and/or the simulated human resource plan 70 is input into a pre-calculated lookup table, a neural network, or the like. The optimization processor 56 is programmed to generate random numbers from distributions specified in the data of the set of the third inputs 48 and the simulated human resource plan 70. The optimization processor 56 also includes an optimized plan generator processor 74 programmed to generate an optimized human resource plan 76 reflecting an optimal solution to the multi-goal objective function data while satisfying the data of the regulations and requirements data. The optimized plan generator processor 74 uses metaheuristic methods (e.g., a greedy algorithm, a Tabu search, a genetic algorithm, simulated annealing, and the like) along with inputs from the set of third inputs 50 and the simulated human resource plan 70 to create the optimized human resource plan 76.

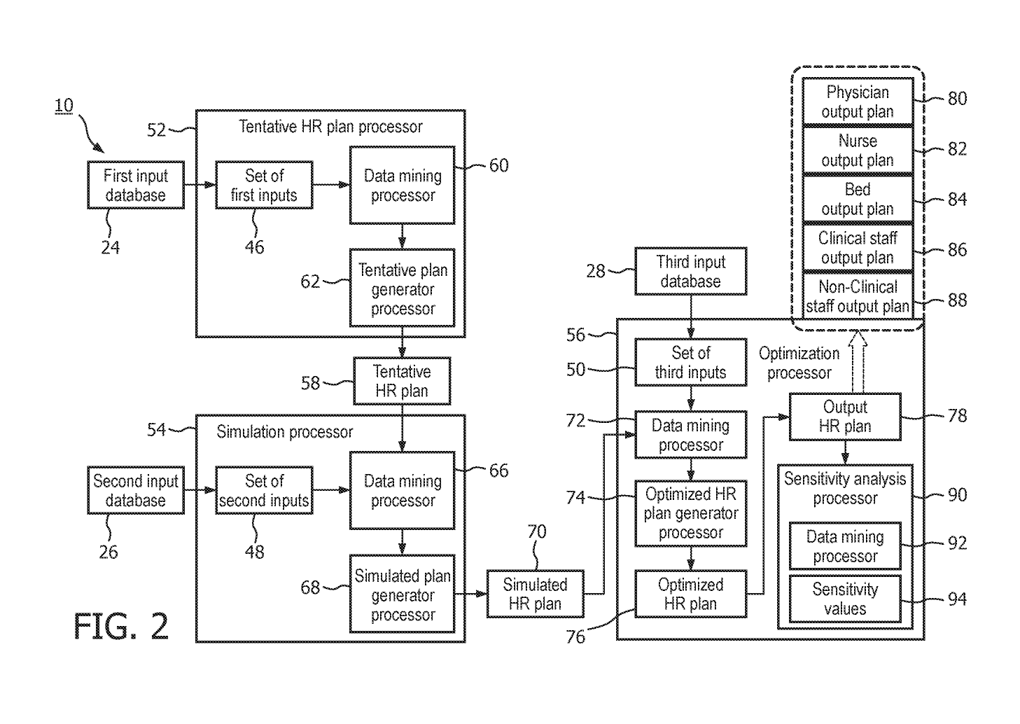

The optimized human resource plan 76 includes one or more output human resource plans 78. The one or more output human resource plans 78 can include data related to one or more hospital resources and/or services. Each of the output human resource plans 78 are specialty-specific plans, which is different based on different specialty units. For example, the one or more output human resource plans 78 of FIG. 2 can include a physician output plan 80, a nurse output plan 82, a hospital bed output plan 84, a clinical support staff output plan 86, and a non-clinical support staff output plan 88. Each of the output human resource plans 78 are based on an optimal FTE number of each of the inputs 46, 48, and 50. The output human resource plans 78 provide an assessment of which hospital resources should allocated to treat the patients admitted to the medical institution.

In one example, the optimization processor 56 includes a sensitivity analysis processor 90 programmed to adjust one parameter of the output human resource plans 78 at a time to see which parameters have the most influences thereon. To do so, the sensitivity analysis processor 90 includes a data mining processor 92 programmed to extract values related to the one or more output human resource plans 78. For example, the one or more output human resource plans 78 are input into a pre-calculated lookup table, a neural network, or the like. The optimization processor 56 is programmed to generate random numbers from distributions specified in the data of one or more output human resource plans 78. The sensitivity analysis processor 90 generates a sensitivity human resource plan report 94 that determines which parameters of the output human resource plans 78 should be adjusted to further optimize the output human resource plans 78. The sensitivity analysis processor 90 uses metaheuristic methods (e.g., a greedy algorithm, simulated annealing, and the like) along with inputs from the one or more output human resource plans 78 to create the output human resource plans 78. In some examples, the optimization processor 56 is programmed to continuously monitor and evaluate the output human resource plans 78. In other examples, the optimization processor 56 is programmed to produce self-effectiveness evaluation updates of the output human resource plans 78.

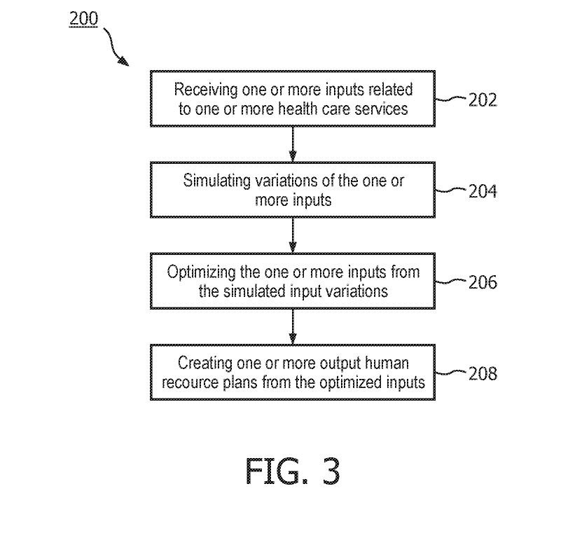

With reference to FIG. 3, a method 200 for creating a human resources plan for a hospital system is provided. At Step 202, one or more inputs 46, 48, 50 related to one or more health care services that are each associated with at least one of hospital data and target data are received. At Step 204, variations of the one or more inputs 46, 48, 50 are simulated. At Step 206, the one or more inputs 46, 48, 50 are optimized from the simulated input variations. At Step 208, one or more output human resource plans 78 are created from the optimized inputs.

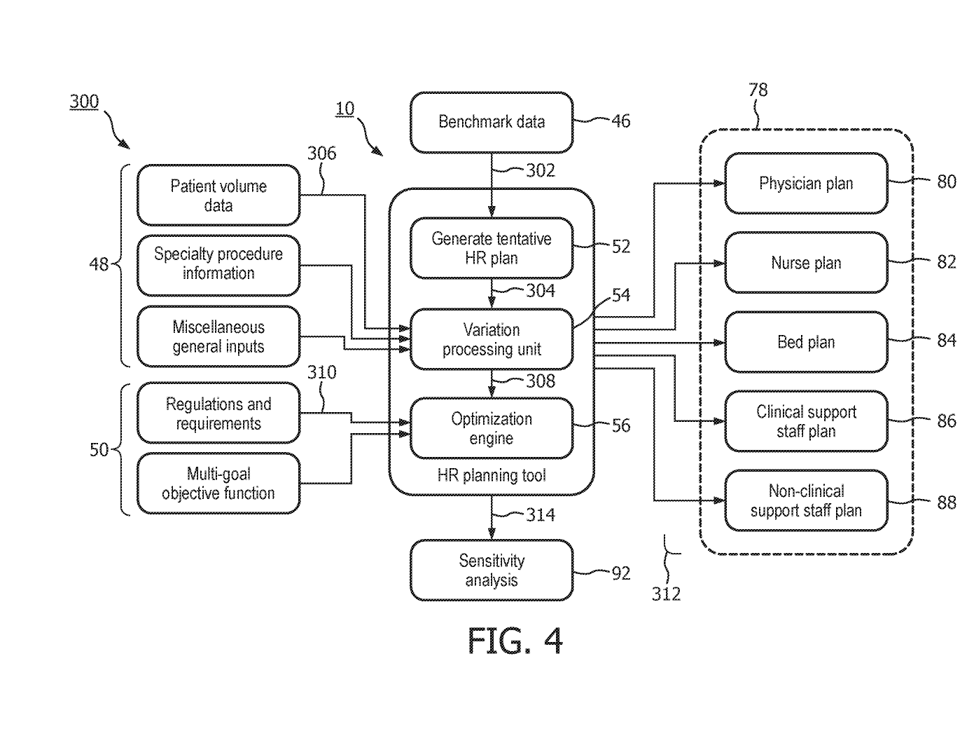

With reference to FIG. 4, a method 300 is provided for creating a human resources plan is provided. At Step 302, the tentative human resource plan processor 52 receives the set of first inputs (i.e. benchmarks) 46. At Step 304, the tentative human resource plan processor 52 generates the tentative human resource plan 58 based on the set of first inputs 46 (see also FIG. 2). At Step 306, the simulation processor 54 receives the tentative human resource plan 58 and a set of second inputs 48. At Step 308, the simulation processor 54 generates a simulated human resource plan 70 (see also FIG. 2). At Step 310, an optimization processor 56 receives the simulated human resource plan 70 and the set of third inputs 50. As seen in FIG. 4, these inputs 50 include the multi-goal optimization function to be optimized, along with any constraints such as those imposed by governmental regulations, requirements, and the like. At Step 312, the optimization processor 56 generates one or more output human resource plans 78 (see also FIG. 2). For example, the one or more output human resource plans 78 of FIG. 2 can include a physician output plan 80, a nurse output plan 82, a hospital bed output plan 84, a clinical support staff output plan 86, and a non-clinical support staff output plan 88. At Step 314, the optimization processor 56 generates a sensitivity analysis report 92.

### EXAMPLE

With continuing reference to FIG. 4, the input data are provided by the hospital. First the benchmark data are used to generate tentative HR plans. The benchmark data can be a rough range of potential FTE numbers for different types of healthcare employees (e.g., doctors, nurses, clinical staff, non-clinical staff). These FTE ranges will then become input for the simulation processor 54 (along with the patient volume data, specialty procedure information and miscellaneous general inputs) and the optimization processor 56. Most of this data are treated as random variables to capture the variations in the human resources plan system 10. The core of the simulation processor 54 is a simulation model, which can generate random numbers from distributions specified in the input data and provide corresponding statistics for the optimization engine.

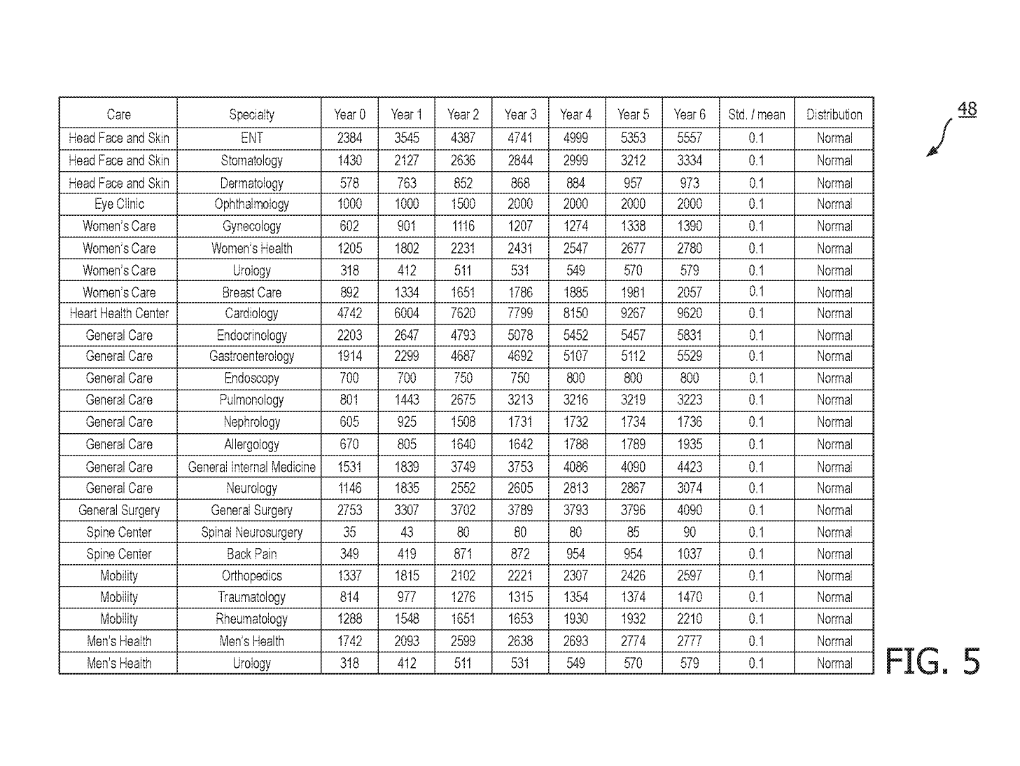

With reference to FIG. 5, an example of one of the inputs 46, 48, and 50 is shown. In this example, the input 48 is the patient volume input data. Besides point estimates of the average yearly patient volume, the coefficient of variance (standard deviation divided by mean) and the distribution of the patient volume data are also provided. In this dataset, all the patient volumes are assumed to be normally distributed and the coefficient of variance is 0.1. This is due to lack of historical data and thus the prediction is quite vague. The data includes a normal distribution. More generally, the human resource planning system 10 can provide other kinds of random distributions, including random distributions described by more or different parameters than mean and standard deviation.

The specialty procedure information includes data that might vary among different specialty units (e.g., patient length of stay, percentage of ambulatory visits that need surgery and the corresponding ambulatory surgery time, patient to nurse ratio, and the like). Most of the specialty based input data are also treated as random variables similar to the patient volume data in the simulation processor 54. The simulation processor 54 generates random numbers according to the distributions specified in the input data 48. Miscellaneous general inputs contain information that is same for all specialty units in the hospital, such as working hours per day, working days within a year, percentage of patient related activity, and the like.

After the simulation processor 54 generates the simulated human resources plan 70, the optimization processor 56 generates one or more optimal human resource plans 76. The regulations and requirements and multi-goal objective function modules (i.e., the third set of inputs 50) are used to build the optimized human resource plans 76. The requirements could, for example, include minimum coverage rate (percentage of days when patient demand can be fully covered in regular working hours), range of resource utilities, maximum overtime, and constraints on yearly variation of physician and nurse FTEs. The multi-goal objective function is formulated as the sum of several different terms with their corresponding weights of importance. For example, the weighted sum of total FTE number and average overtime can be minimized.

In the optimization processor 56, various algorithms can be used to solve the human resource planning problem. As just one illustrative example, a fast initial solution could be based on a greedy search algorithm. In most cases there is limited number of constraints (e.g. the governmental regulations and requirements). Hence the objective function is typically a unimodal function of the FTE number, while the fast initial solution is equivalent to the global optimal solution. Other optimization approaches, such as heuristic algorithms, e.g. Tabu search, simulated annealing, and genetic algorithm, can be employed to solve the problem.

Referring back to FIG. 4, the output patient care plans 78 are plans for different resources, including a physicians plan 80, a nurses plan 82, a beds plan 84, a clinical support staff plan 86, and a non-clinical support staff 88. All the staffing and bed plans are specialty-specific plans, which is different based on different specialty units. It is noted that in the illustrative embodiments patient beds are treated as a human resources specialty unit. This is a convenient mechanism because, although patient beds are technically not a “human resource”, they are so closely tied to human resources planning that they are advantageously treated in the illustrative human resources planning techniques as a “human resource” specialty unit. This allows the number of patient beds to be optimized along with the human resource staffing levels.

It is also noted that the specialty units can be variously defined, for example with respect to medical training (or lack thereof), e.g. physician, nurse, and non-clinical specialty units; and/or by clinical care area, e.g. the physicians can be divided into a cardiologist specialty unit, a pediatrician specialty unit, and so forth. Similarly, it is contemplated for there to be various different patient bed specialty units, e.g. a cardiac care beds specialty unit, a pediatric care beds specialty unit, and so forth.

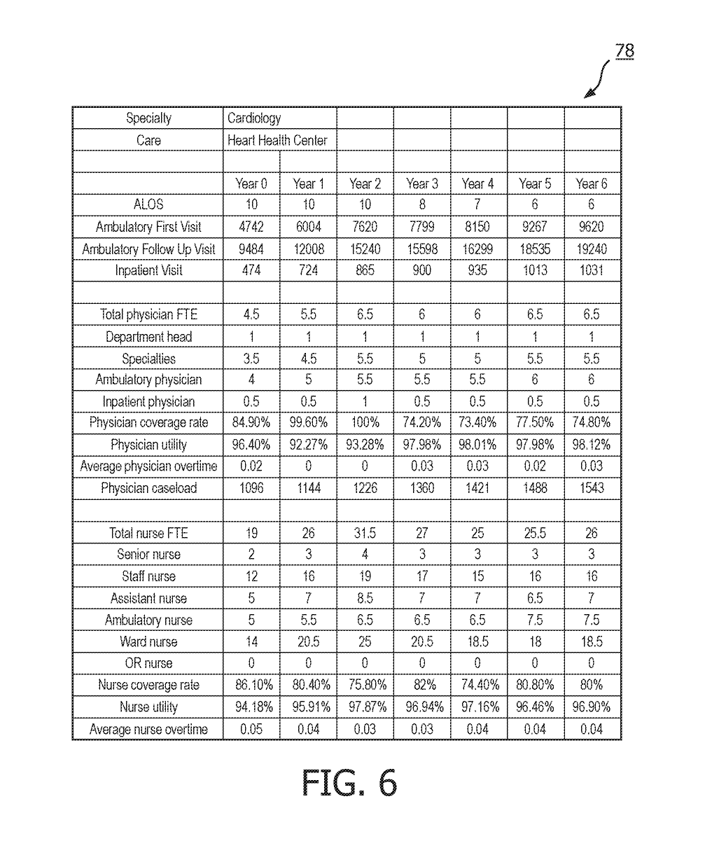

With reference to FIG. 6, a sample output human resource plan 78 for cardiology physicians and nurses is shown. Information related to department head and specialties, ambulatory physician and inpatient physician are also shown. The system 10 can also provide physician coverage rate, utility, average overtime, and yearly caseload generated from the simulation model based on the total physician FTE number. With this data, healthcare consultants or hospital administrators have a clear idea of what is expected to happen if they apply this human resource plan 78.

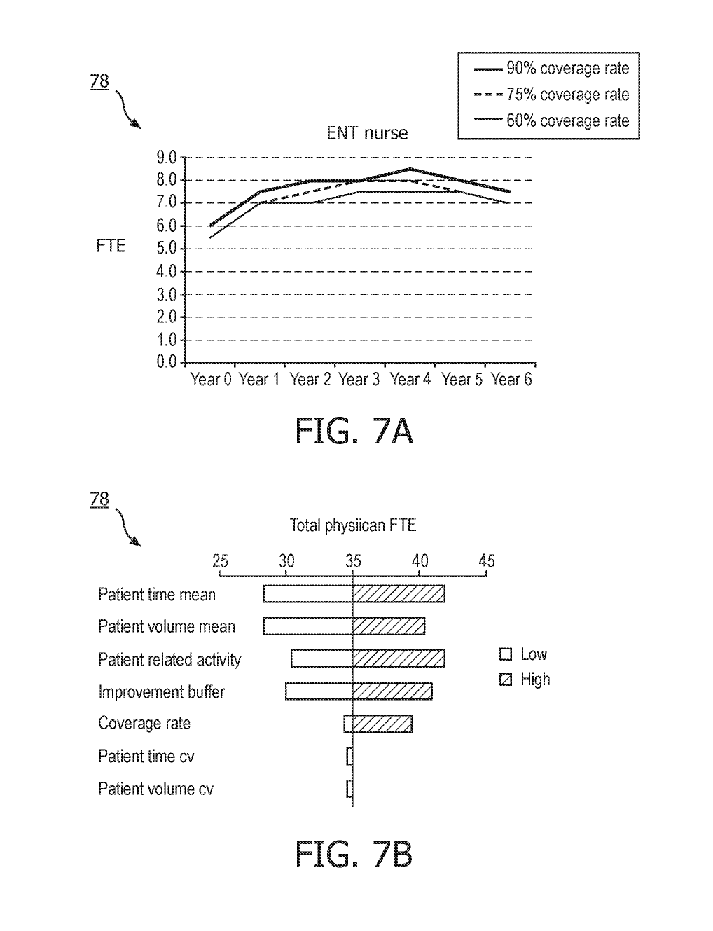

With reference to FIG. 7A, a sample output 78 for emergency nursing triage (ENT) nurses with different coverage rate is shown. In this example, the nurse FTE number increases as the required coverage rate goes up. This data can also be obtained from the human resource planning system by adjusting the coverage rate parameter. Besides the major outputs of the planning for different types of healthcare resources, the system 10 can also provide the sensitivity analysis report 92 for various input parameters. One parameter is adjusted at a time to see the change in the human resource plan 78. The sensitivity analysis processor 90 can allow healthcare consultants or hospital administrators to have an idea of which parameters are more important and should be carefully adjusted. With reference to FIG. 7B, an output plan 78, shown as a sample tornado chart depicting influences of several different input parameters on total physician FTE, is shown. For this particular example, the average patient volume and average patient time have the most influenceon the total physician FTE number.

As used herein, a memory includes one or more of a non-transient computer readable medium; a magnetic disk or other magnetic storage medium; an optical disk or other optical storage medium; a random access memory (RAM), read-only memory (ROM), or other electronic memory device or chip or set of operatively interconnected chips; an Internet/Intranet server from which the stored instructions may be retrieved via the Internet/Intranet or a local area network; or so forth. Further, as used herein, a processor includes one or more of a microprocessor, a microcontroller, a graphic processing unit (GPU), an application-specific integrated circuit (ASIC), a field-programmable gate array (FPGA), or so forth. A user input device includes one or more of a mouse, a keyboard, a touch screen display, one or more buttons, one or more switches, one or more toggles, and the like; and a display device includes one or more of a LCD display, an LED display, a plasma display, a projection display, a touch screen display, and the like. Stated another way, the human resource plan system 10 can be a non-transitory computer readable medium carrying software to control a processor.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A human resources (HR) planning system (10) comprising:
an electronic processor (40) programmed to perform a HR planning method including:
generating a tentative HR plan (58) based on received parameters including at least patient volume parameters and staffing parameters for a plurality of HR specialty units;
computing a simulated HR plan (70) from the tentative HR plan based on received parameter variability data, the simulated HR plan representing parameters of the tentative HR plan as random variables with distributions representing the parameter variability;
optimizing the random variables of the simulated HR plan with respect to an objective function (50) representing objectives for staffing of the medical institution; and
outputting staffing plans for the HR specialty units wherein the staffing plans are determined from the optimized random variables representing the staffing parameters in the optimized simulated HR plan.

2. The HR planning system (10) of claim 1 wherein the HR specialty units include at least one physician specialty unit, at least one nurse specialty unit, and at least one non-clinical staff specialty unit.

3. The HR planning system (10) of claim 2 wherein the medical specialties further include at least one patient beds specialty unit.

4. The HR planning system (10) of any one of claims 1-3 wherein the staffing parameters are represented as full-time equivalent (FTE) values in the tentative HR plan (58).

5. The HR planning system (10) of any one of claims 1-4 wherein the optimizing comprises performing a constrained optimization including a constraint defined by a governmental regulation.

6. The HR planning system (10) of any one of claims 1-5 wherein the optimizing is performed using at least one of: a greedy search algorithm, a Tabu search, simulated annealing, and a genetic algorithm.

7. The HR planning system (10) of any one of claims 1-6 further comprising:
performing sensitivity analysis on parameters of the optimized HR plan (76) represented as random variables;
wherein the outputting includes displaying sensitivity of the staffing plans as determined by the sensitivity analysis.

8. The HR planning system (10) of claim 7 wherein performing sensitivity analysis comprises:
adjust a parameter individual and assessing effect of the adjustment on the staffing plans.

9. A non-transitory storage medium storing instructions readable and executable by an electronic processor (40) to perform a human resources (HR) planning method comprising:
generating a tentative HR plan (58) based on received parameters including patient volume parameters and staffing parameters for a plurality of specialty units defined at least by medical expertise into physician, nursing, and non-clinical support staff specialty units;
computing a simulated HR plan (70) from the tentative HR plan based on received parameter variability data, the simulated HR plan representing parameters of the tentative HR plan as random variables with distributions representing the parameter variability;
performing a constrained optimization of the random variables of the simulated HR plan with respect to an objective function (50) representing objectives for staffing of the medical institution and constraints defined at least by governmental regulations; and
outputting staffing plans for the specialty units wherein the staffing plans are determined from the optimized random variables representing the staffing parameters in the optimized simulated HR plan.

10. The non-transitory storage medium of claim 9 wherein the specialty units are further defined by clinical care area.

11. The non-transitory storage medium of any one of claims 9-10 wherein the specialty units further include patient bed specialty units.

12. The non-transitory storage medium of any one of claims 9-11 wherein the staffing parameters are represented as full-time equivalent (FTE) values in the tentative HR plan (58).

13. The non-transitory storage medium of any one of claims 9-12 wherein the constrained optimization is performed using at least one of: a greedy search algorithm, a Tabu search, simulated annealing, and a genetic algorithm.

14. The non-transitory storage medium of any one of claims 9-13 further comprising:
performing sensitivity analysis on parameters of the optimized HR plan (76) represented as random variables;
wherein the outputting includes displaying sensitivity of the staffing plans as determined by the sensitivity analysis.

15. A method for creating a human resources plan for a hospital system, the method including:
receiving, at an electronic processor (40), one or more inputs (46, 48, 50) related to one or more health care services that are each associated with at least one of hospital data and target data;
simulating variations of the one or more inputs (46, 48, 50);
optimizing the one or more inputs (46, 48, 50) from the simulated input variations; and
creating one or more output human resource plans (78) from the optimized inputs;
wherein the simulating, the optimizing, and the creating are performed by the electronic processor.

16. The method according to claim 15 further including:
performing a sensitivity analysis by adjusting the one or more inputs (46, 48, 50) to determine which input most influences the one or more output health resource plans (78).

17. The method according to any one of claims 15-16 wherein the one or more inputs include:
a set of first inputs (46) related to benchmark data;
a set of second inputs (48) related to patient volume data, specialty procedure information data, and miscellaneous general data; and
a set of third inputs (50) related to regulations and requirements data and multi-goal objective function data.

18. The method according to claim 17 wherein:
the tentative human resources plan (58) is generated from the set of first inputs (46);
the simulated human resources plans (70) are generated from the set of second inputs (48) and the tentative human resources plan (58); and
one or more optimized human resources plans (76) are generated based on the set of third inputs (50) and the simulated human resources plans (70).

19. The method according to any one of claims 15-18, wherein simulating variations of the one or more inputs (46, 48, 50) from at least one of hospital data and target data related thereto further includes:
generating one or more optimized human resources plans (76) based on random numbers from distributions specified in the one or more inputs (46, 48, 50).

20. The method according to any one of claims 15-19, wherein the one or more output human resource plans (78) include one or more of a physician plan (80), a nurse plan (82), a bed plan (84), a clinical support staff plan (86), and a non-clinical support staff plan (88).
